(19) United States
(12) Patent Application Publication
(10) Pub. No.: US 2023/0191157 A1
(43) Pub. Date: Jun. 22, 2023

(54) Automatic estimation of positions of brachytherapy seeds

(71) Applicant: Alpha Tau Medical Ltd., Jerusalem (IL)

(72) Inventors: Ilay Kamai, Kibbutz Tuval (IL); Ronen Segal, Jerusalem (IL); Yadin Cohen, Jerusalem (IL); Amnon Gat, Matan (IL)

(21) Appl. No.: 18/071,687

(22) Filed: Nov. 30, 2022

Related U.S. Application Data
(60) Provisional application No. 63/291,966, filed on Dec. 21, 2021.

(51) Int. Cl. A61N 5/10; G06T 19/20; G06T 15/08; G06T 7/73; G06V 20/69; G06V 10/762; G16H 30/40

(52) U.S. Cl. CPC A61N 5/1039; G06T 7/74; G06T 15/08; G06T 19/20; G06V 10/762; G06V 20/695; G16H 30/40; A61N 2005/1024; G06T 2200/24; G06T 2207/30096; G06T 2219/2004; G06V 2201/034

(57) ABSTRACT

A system includes a memory, configured to store program instructions, and a processor. The processor is configured to load the program instructions from the memory, and by executing the program instructions, to process a three-dimensional image of a portion of a body of a subject in which multiple brachytherapy to identify clusters of voxels of the image corresponding to the seed groups, respectively, to compute respective estimated positions of the brachytherapy seeds based on respective dimensions of each of the clusters, and to store or communicate the estimated positions for use in computing an effective dose of the brachytherapy seeds. Other embodiments are also described.

### CROSS-REFERENCE TO RELATED APPLICATIONS

The present application claims the benefit of US Provisional Application 63/291,966, filed Dec. 21, 2021, whose disclosure is incorporated herein by reference.

### FIELD OF THE INVENTION

The present application relates generally to brachytherapy, and particularly to automated techniques for facilitating a .

### BACKGROUND

In a brachytherapy procedure, one or more brachytherapy seeds are implanted, e.g., using a needle, near a tumor in a subject. The seeds, which may be metallic or metallically-coated for example, carry radionuclide atoms that emit destructive radiation at the tumor.

### SUMMARY OF THE INVENTION

There is provided, in accordance with some embodiments of the present invention, a system including a memory, configured to store program instructions, and a processor. The processor is configured to load the program instructions from the memory, and by executing the program instructions, to process a three-dimensional image of a portion of a body of a subject in which multiple brachytherapy seeds grouped into one or more seed groups are implanted, so as to identify clusters of voxels of the image corresponding to the seed groups, respectively, to compute respective estimated positions of the brachytherapy seeds based on respective dimensions of each of the clusters, and to store or communicate the estimated positions for use in computing an effective dose of the brachytherapy seeds.

There is further provided, in accordance with some embodiments of the present invention, a method including processing a three-dimensional image of a portion of a body of a subject in which multiple brachytherapy seeds grouped into one or more seed groups are implanted, so as to identify clusters of voxels of the image corresponding to the seed groups, respectively. The method further includes computing respective estimated positions of the brachytherapy seeds based on respective dimensions of each of the clusters, and storing or communicating the estimated positions for use in computing an effective dose of the brachytherapy seeds.

In some embodiments, the method further includes:
displaying at least part of the image with overlaid markers at the estimated positions so as to allow a user to adjust the estimated positions by performing an action selected from the group of actions consisting of: overlaying one or more additional markers, deleting one or more of the markers, and moving one or more of the markers; and
computing the effective dose based on the adjusted estimated positions.

In some embodiments, processing the image includes processing the image by applying a Bayesian Gaussian mixture model to the image.

In some embodiments, processing the image includes processing the image by applying a connected-components clustering algorithm to the image .

In some embodiments, computing the respective estimated positions of the brachytherapy seeds includes:
based on the respective dimensions of the clusters, computing respective estimated numbers of the brachytherapy seeds in the seed groups; and
computing the respective estimated positions based on the respective estimated numbers .

In some embodiments, for each of the clusters, computing the estimated of the brachytherapy seeds in the seed number of those of group corresponding to the cluster includes:
computing a length of a main axis of the cluster; and
of seeds in computing the estimated number the brachytherapy the seed group corresponding to the cluster based on the length.

In some embodiments, for each of the seed groups, computing the respective estimated positions of those of the brachytherapy seeds in the seed group includes computing the estimated positions such that, per the estimated positions, respective sub-clusters of the voxels corresponding to those of the brachytherapy seeds in the seed group are distributed uniformly along the main axis.

In some embodiments, for each of the seed groups, computing the respective estimated positions of those of the brachytherapy seeds in the seed group includes:
segmenting the main axis into length segments; and
computing the estimated positions such that, per the estimated positions, the sub-clusters are aligned with the main axis and centered on the segments, respectively.

In some embodiments, computing the estimated positions includes computing the estimated positions by computing respective estimated center coordinates and estimated orientation vectors of the brachytherapy seeds.

In some embodiments, computing the respective estimated positions of the brachytherapy seeds includes:
receiving a total number of the brachytherapy seeds from a user; and
computing the respective estimated positions of the brachytherapy seeds based on the total number.

There is further provided, in accordance with some embodiments of the present invention, a computer software product including a tangible non-transitory computer-readable medium in which program instructions are stored. The instructions, when read by a processor, cause the processor to process a three-dimensional image of a portion of a body of a subject in which multiple brachytherapy seeds grouped into one or more seed groups are implanted, so as to identify clusters of voxels of the image corresponding to the seed groups, respectively. The instructions further cause the processor to compute respective estimated positions of the brachytherapy seeds based on respective dimensions of each of the clusters, and to store or communicate the estimated positions for use in computing an effective dose of the brachytherapy seeds.

The present invention will be more fully understood from the following detailed description of embodiments thereof, taken together with the drawings, in which:

### BRIEF DESCRIPTION OF THE DRAWINGS

FIG. 1 is a schematic illustration of a system for computing an effective brachytherapy dose, in accordance with some embodiments of the present invention;

FIG. 2 is a flow diagram for an example algorithm for estimating positions of brachytherapy seeds, in accordance with some embodiments of the present invention; and

FIG. 3 is a schematic illustration of a technique for estimating positions of brachytherapy seeds in a seed group based dimensionsof a corresponding voxel cluster, in accordance with some embodiments of the present invention.

### DETAILED DESCRIPTION OF EMBODIMENTS

### Overview

After an implantation of brachytherapy seeds, an image of the implantation site is acquired. A physician (or another user) then marks the image so as to indicate the position of each seed. Based on the marked positions, a computer calculates the effective dose of the seeds, e.g., using the American Association of Physicists in Medicine (AAPM) Task Group No. 43 (TG-43) formalism. This calculation may help the physician decide whether there is a need to move the seeds and/or implant additional seeds.

In some cases, however, the seeds are implanted in groups, referred to herein as “seed groups,” and the image does not clearly delineate between the individual seeds in a seed group. Moreover, although the user may know the total number of implanted seeds, the user may not necessarily know the number of seeds in each seed group. Furthermore, the image may not clearly delineate between adjacent seed groups. In such cases, marking each individual seed may be difficult and time-consuming.

To address this challenge, embodiments of the present invention provide a system and method for automatically estimating the positions of the seeds. Following the estimation, markers are overlaid on the image so as to indicate the estimated positions. Thus, the user need not mark the image from scratch; rather, the user may simply adjust the estimated positions by adding, deleting, and/or shifting markers.

Cleverly, rather than attempting to automatically identify each individual seed in the image (which might be prohibitively difficult), embodiments of the present invention identify clusters of voxels corresponding to the seed groups (i.e., representing the seed groups in the image), and then estimate the positions of the seeds based on the respective dimensions of each of the clusters. seeds based on the respective dimensions of each of the clusters. Advantageously, this estimation may capitalize on prior knowledge of the shape and size of each seed and the manner in which the seeds were implanted.

For example, each seed may have a longitudinal (e.g ., cylindrical) shape, and the seeds may be lined up end-to-end in the implantation needle. In view of this prior knowledge, each seed group may be assumed to contain a linear arrangement of seeds, such that the number of seeds in the seed group may be accurately estimated by dividing the length of the main axis of the cluster corresponding to the seed group by the known length of each seed. The seeds may then be assumed to be distributed uniformly along the main axis.

In some cases, a first clustering algorithm applied to the image may erroneously return a single cluster corresponding to multiple adjacent seed groups. In such cases, the system may reject the cluster — i.e., refrain from estimating a number of seeds for the cluster — in response to the abnormally large size of the cluster. The system may then apply another clustering algorithm to the image, in an attempt to differentiate between the adjacent seed groups. For example, after applying a Bayesian Gaussian mixture model to the image, the system may apply a connected-components clustering algorithm to the image.

### System Description

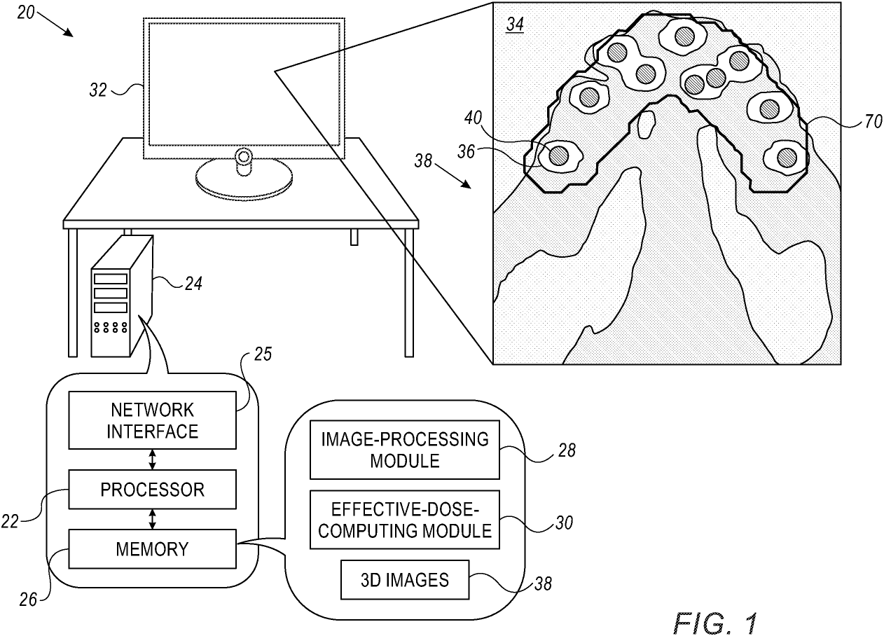

Reference is initially made to FIG. 1, which is a schematic illustration of a system 20 for computing an effective brachytherapy close, in accordance with some embodiments of the present invention.

System 20 comprises a processor 22, which may belong to a standard desktop computer 24, a laptop computer, a smartphone, a medical computer, a cloud server, or any other computing device. System 20 further comprises a memory 26, comprising a volatile memory, such as a Random Access Memory (RAM), and/or a non-volatile memory, such as a flash drive. Memory 26 may be located, in its entirety, on the same computing device as processor 22; alternatively, at least part of the memory may be located remotely from the processor.

Memory 26 is configured to store data, including, for example, three-dimensional (3D) medical images 38 of a portion of a body of a subject. Images 38 may be acquired using any suitable modality such as computational tomography (CT), magnetic resonance imaging (MRI), or ultrasound.

Typically, system 20 further comprises a network interface 25 comprising, for example, a network interface controller (NIC). Processor 22 is configured to exchange communication over a computer network, such as the Internet, via network interface 25.

System 20 further comprises a display 32, on which processor 22 may display any suitable data or output. For example, as shown in FIG. 1, the processor may display slices 34 of an image 38 on display 32. (Multiple slices of the image may be displayed simultaneously.) Each slice 34 may show the anatomy of the portion of the body of the subject, along with implanted seed groups 36 of brachytherapy seeds.

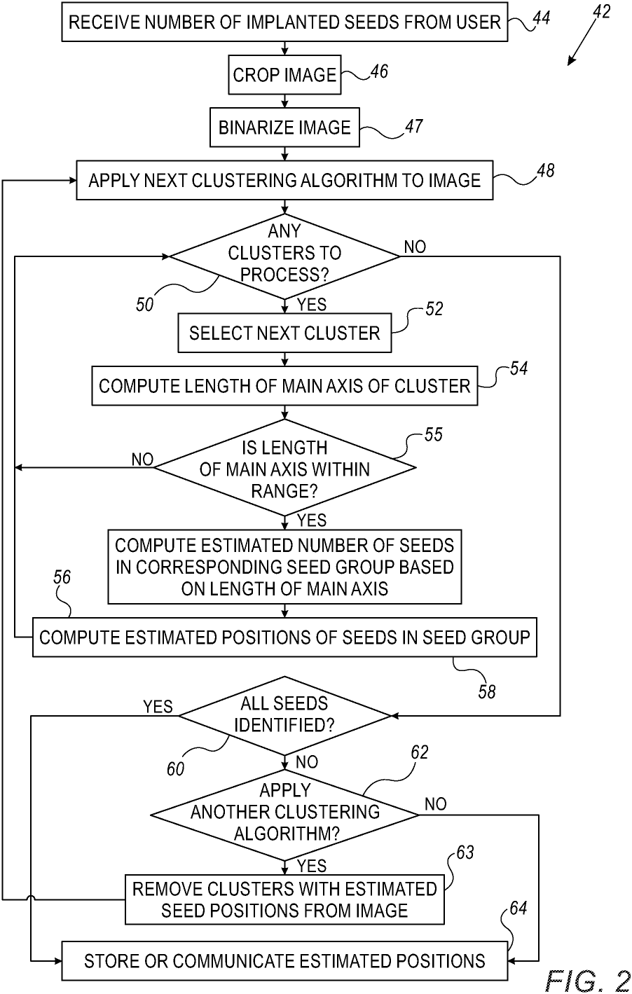

Due to the physical properties of the brachytherapy seeds, seed groups 36 contrast with the surrounding anatomy. For example, in a CT image, seed groups 36 are typically brighter than the surrounding anatomy. (For example, as shown in FIG. 1, which assumes slice 34 belongs to a CT image, seed groups 36 may be white.) However, due to the proximity of the seed groups to each other, advanced image-processing techniques, e.g., as described below with reference to FIG. 2, may be required to delineate between the individual seed groups.

In some embodiments, display 32 comprises a touch screen. Alternatively or additionally, system 20 may further comprise a keyboard, a mouse, and/or any other input interface. The input interfaces may be used, by a user, to provide the processor with the various inputs described herein.

Memory 26 is further configured to store program instructions for processing image 38 so as to facilitate computing an effective dose of the brachytherapy seeds based on respective estimated positions of the brachytherapy seeds. Optionally, memory 26 may further store program instructions for computing the effective dose. Processor 22 is configured to load the instructions from the memory and to execute the instructions.

Typically, the program instructions are grouped into modules. For example, memory 26 may store an image-processing module 28, which includes instructions for processing image 38, and an effective-dose-computing module 30, which includes instructions for computing the effective dose.

To facilitate computing an effective dose, processor 22 first computes respective estimated positions of the brachytherapy seeds in seed groups 36, as described in detail below with reference to the subsequent figures. Subsequently, the processor stores or communicates these estimated positions for use in computing the estimated dose of the seeds.

For example, for embodiments in which the processor computes the effective close, the processor may store the estimated positions in memory 26. Subsequently, the processor may load the estimated positions from the memory, and then display at least part of the image with overlaid markers 40 at the estimated positions. For example, as shown in FIG. 1, the processor may display one or more slices of the image with overlaid markers 40. Alternatively, the processor may display a three-dimensional rendering of the image with the overlaid markers. A user may then adjust the estimated positions by overlaying one or more additonal markers, deleting one or more of the markers, and/or moving one or more of the markers. Subsequently, the processor may compute the effective dose based on the adjusted estimated positions.

Alternatively, for embodiments in which another processor computes the effective dose, processor 22 may communicate the estimated positions (e.g., over network interface 25) to the other processor, and the other processor may then execute the functionality described above.

In general, the processor may read the program instructions and data from the memory, and write data to the memory, over any suitable one or more interfaces, including network interface 25 in the event that the memory is located remotely from the processor.

In general, processor 22 may be embodied as a single processor, or as a cooperatively networked or clustered set of processors.

In some embodiments, processor 22 is embodied as a programmed processor comprising, for example, a central processing unit (CPU) and/or a Graphics Processing Unit (GPU). Program instructions, including software program instructions, and/or data may be loaded for execution and processing by the CPU and/or GPU. The program instructions and/or data may be downloaded to the processor in electronic form, over a network, for example. Alternatively or additionally, the program instructions and/or data may be provided and/or stored on non-transitory tangible media, such as magnetic, optical, or electronic memory. Such program instructions and/ or data, when provided to the processor, produce a machine or special-purpose computer, configured to perform the tasks described herein.

Alternatively, the functionality of processor 22 may be implemented in hardware, e.g., using one or more fixed-function or general-purpose integrated circuits, Application-Specific Integrated Circuits (ASICs), and/or Field-Programmable Gate Arrays (FPGAs) .

### Computing Estimated Positions of the Seeds

Reference is now additionally made to FIG. 2, which is a flow diagram for an example algorithm 42 for estimating positions of brachytherapy seeds, in accordance with some embodiments of the present invention.

As described above, processor 22 is configured to compute respective estimated positions of brachytherapy seeds implanted in a subject. This functionality may be performed, for example, by executing algorithm 42.

Algorithm 42 begins with an optional receiving step 44, at which the processor receives the number of implanted seeds from a user. For example the user may use a keyboard to input the number.

As described below, the processor may compute the estimated positions of the seeds based on the received number. For example, the processor may apply successive clustering algorithms until the number of identified seeds is at least equal to the number of implanted seeds. Alternatively or additionally, the number of implanted seeds may be provided as input to a clustering algorithm. Alternatively or additionally, the processor may estimate the positions of the seeds under the constraint that the number identified seeds not exceed the number of implanted seeds.

In some embodiments, before delineating each individual seed group in image 38, the processor crops the image at a cropping step 46. For example, the user may draw a contour 70 around the seed groups in multiple slices of image 38. Subsequently, the processor may define a three-dimensional bounding box that bounds contours 70, and crop the image by removing voxels outside the bounding box.

(Notwithstanding FIG. 1, it is noted that, typically, contours 70 are not displayed after the positions of the seeds have been estimated and markers 40 have been overlaid onto the image . )

In some embodiments, the processor delineates between the seed groups by applying one or more clustering algorithms to the image.

In some such embodiments, the processor first binarizes the image (e.g., the cropped image) at a binarizing step 47. Following binarizing step 47, each voxel in the image has a value of zero or one, depending on whether the voxel is presumed to belong to a seed group. For example, voxels presumed to belong to a seed group may have a value of one, and all other voxels may have a value of zer o.

To perform this binarization, the processor applies a threshold to the image. For imaging modalities in which seed groups 36 are brighter than the surrounding tissue, any voxels having a value greater than the threshold are presumed to belong to a seed group. For other imaging modalities, any voxels having a value less than the threshold arepresumed to belong to a seed group.

The processor may calculate the aforementioned threshold by applying any suitable equation. For example, for a CT image in which the seed groups are brighter than the surrounding tissue, the equation may be:
T = max(X, max(I) - Z), where:
T is the threshold in Hounsfield units,
max(I) is the maximum voxel value, in Hounsfield units, in the image I, and
X and Z are predefined constants, in Hounsfield units, derived from the statistics of multiple CT images of implanted brachytherapy seeds. For example, X may be between 1500 and 2600, such as between 1800 and 2200, and/or Z may be between1500 and 2000, such as between 1700 and 1900.

Subsequently to binarizing the image (or if binarizing step 47 is omitted), the processor applies a clustering algorithm to the image at a clustering step 48. The clustering algorithm identifies clusters of voxels in the image, each clusterof voxels potentially corresponding to a seed group. Some example clustering algorithms, each of which may be applied to the image, are described further below.

Subsequently to clustering step 48, the processor checks, at a checking step 50, whether the clustering algorithm returned any clusters of voxels. If yes, the processor selects a cluster at a cluster-selecting step 52. Subsequently, the processor ascertains whether the cluster corresponds, at least approximately, to a single seed group. If yes, the processor computes an estimated number of seeds in the seed group.

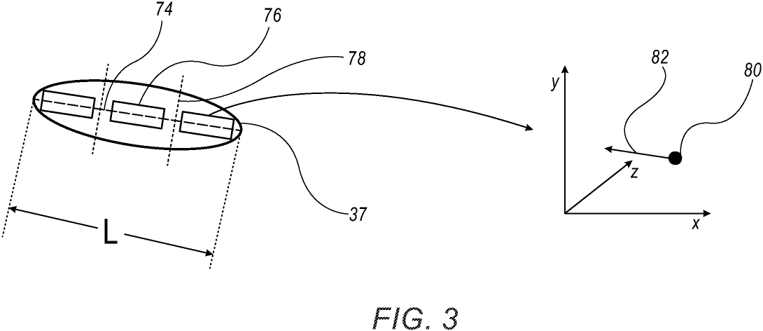

In this regard, reference is now additionally made to FIG. 3, which is a schematic illustration of a technique for estimating positions of brachytherapy seeds in a seed group based on dimensionsof a corresponding voxel cluster 37, in accordance with some embodiments of the present invention. (For ease of illustration, cluster 37 is drawn in two dimensions.)

Typically, the processor computes the respective estimated numbers of the brachytherapy seeds in the seed groups based on the respective dimensions of the voxel clusters, and computes the estimated positions of the seeds based on the respective estimated numbers.

For example, in some embodiments, subsequently to selecting cluster 37 at selecting step 52, the processor computes, at a computing step 54, the length L of the main axis 74 of the cluster. (In the context of the present application, including the claims, the “main axis” of a cluster is the longest hypothetical line having endpoints on the perimeter of the cluster and passing through the cluster.) Subsequently, at a checking step 55, the processor checks whether L is within a predefined range. For example, after converting L (based on the resolution of the image) from a number of pixels to a suitable unit of length such as mm, the processor may check whether L is between b*s and (1-b+m)*s, where:
s is the length of each seed (e.g., 10 mm),
m is the maximum number of seeds that may be delivered in the needle used for implantation (e.g., six), and hence the maximum number of seeds in a seed group, and
b is a predefined constant between 0.5 and 1, such as 0.5.

(Alternatively to converting L to a unit of length, the processor may convert s to a number of pixels.)

If L is not within the predefined range, it may be assumed that the cluster does not correspond to a single seed group. In particular, if L is too small, it may be assumed that the cluster corresponds only to part of a seed group. If L is too large, it may be assumed that the cluster corresponds to multiple seed groups or to bone, which may appear similar to brachytherapy seeds. Hence, if L is not within the predefined range, the processor returns to checking step 50.

On the other hand, if L is within the predefined range, it may be assumed that the cluster corresponds to a single seed group. Hence, the processor, at another computing step 56, computes the estimated number N of brachytherapy seeds in the seed group based on length L. For example, the processor may compute N as L/s rounded to the nearest integer.

In other embodiments, at checking step 55, the processor checks whether L/s is within a predefined range. For example, the processor may check whether L/s is between b and (1-b+m). If yes, the processor computes N, e.g., as L/s rounded to the nearest integer. Alternatively, the processor may first compute N, e.g., as L/s rounded to the nearest integer, and then check whether N is greater than zero and is also less than or equal to m.

Subsequently to computing N, the processor computes the estimated positions of the seeds seeds in the seed group corresponding to the cluster, at another computing step 58. For example, the processor may compute the estimated positions such that, per the estimated positions, the sub-clusters 76 of voxels corresponding to the seeds are distributed uniformly along main axis 74. As a specific example, the processor may segment main axis 74 into N equal-length segments, as indicated in FIG. 3 by segmenting indicators 78. Next, the processor may compute the estimated positions such that, per the estimated positions, sub-clusters 76 are aligned with main axis 74 and centered (e.g., both radially and longitudinally) on the segments, respectively.

Typically, the seeds are radially symmetric, such that the processor may compute the estimated positions by computing respective estimated center coordinates 80 and estimated orientation vectors 82 of the seeds. In other words, given that the seeds are radially symmetric, estimated center coordinates 80 and estimated orientation vectors 82 fully describe the estimated positions. For example, for embodiments in which the estimated positions are computed by segmenting main axis 74 as described above, the processor may compute the estimated positions by computing the center points of the segments and the unit vector of main axis 74. By way of illustration, FIG. 3 shows an estimated position of a seed relative to a three-dimensional xyz coordinate system.

Alternatively, the estimated positions may be represented by any other set of coordinates and/or vectors.

Subsequently to computing the estimated positions, the processor returns to checking step 50.

Upon ascertaining, at checking step 50, that no clusters remain to be processed (or that no clusters were identified by the clustering algorithm), the processor checks, at another checking step 60, whether all the seeds were identified, by comparing the total number of identified seeds with the total number of implanted seeds received at receiving step 44. If not all the seeds were identified, the processor checks, at another checking step 62, whether another clustering algorithm is to be applied.

If another clustering algorithm is to be applied, the processor, at a cluster-removing step 63, removes, from the image, any clusters for which seed positions were estimated. For example, if the seed groups are represented by ones seeds in the binarized image, the processor may set the voxels in these clusters to zero. Subsequently, the processor returns to clustering step 48.

On the other hand, if no clustering algorithms remain, or if all the seeds were identified, the processor stores or communicates the estimated positions at a storing-or-communicating step 64. For example, the processor may store or communicate the estimated center coordinates and orientation vectors of the seeds. (In the event that the image was cropped, storing-or-communicating step 64 may include converting the estimated positions from the coordinate system of the cropped image to the original coordinate system of the image.)

In the event that the number of identified seeds is more or less than the number of implanted seeds, the processor may output a warning, e.g., by displaying the warning on display 32. In response to the warning, the user may add or remove markers 40 such that the number of markers equals the number of implanted seeds.

In some embodiments, the processor does not allow the number of identified seeds to be greater than the number of implanted seeds. Rather, the processor estimates the number of seeds in each seed group under the constraint that the total estimated number not exceed the number of implanted seeds.

In alternative embodiments, the processor applies only a single clustering algorithm, regardless of how manyseeds are identified.

### Example Clustering Algorithms

As described above, the processor may apply one ormore clustering algorithms to image 38. Each clustering algorithm is configured to return one or more clusters of voxels corresponding to respective seed group 36.

For embodiments in which the image is binarized prior to the application of any clustering algorithms, the voxels are clustered based on their positions in the image. For embodiments in which the image is not binarized, the voxels may be clustered based on the image is not binarized, the voxels may be clustered based on their positions and also based on their values.

In some embodiments, the clustering algorithms include a Bayesian Gaussian mixture model. In embodiments in which the image is binarized, the features for the model are the coordinates of each voxel. In other embodiments, the features may additionally include the voxel values and/or any function thereof, such as gradients of voxel values. The model may be initialized with the number of implanted seeds (received at receiving step 44) and a type of prior distribution. The model outputs a number of seed groups and an assignment of voxels to seed groups.

In some such embodiments, the type of prior distribution is a Dirichlet distribution or Dirichlet process. The order of the Dirichlet distribution may be a fraction of the number of implanted seeds, such as one third, half, or two thirds of this number.

Alternatively or additionally, the clustering algorithms may include a connected-components clustering algorithm (with a six-neighbor kernel for 3D clustering). For example, the connected-components clustering algorithm may be applied after the Bayesian mixture model, in the event that the latter does not produce a sufficient number of identified seeds.

Alternatively or additionally, the clustering algorithms may include a neural-network clustering algorithm, which utilizes a neural network.

It will be appreciated by persons skilled in the art that the present invention is not limited to what has been particularly shown and described hereinabove. Rather, the scope of embodiments of the present invention includes both combinations and subcombinations of the various features described hereinabove, as well as variations and modifications thereof that are not in the prior art, which would occur to persons skilled in the art upon reading the foregoing description. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system, comprising:
a memory, configured to store program instructions; and
a processor, configured to:
load the program instructions from the memory, and
by executing the program instructions:
process a three-dimensional image of a portion of a body of a subject in which multiple brachytherapy seeds grouped into one or more seed groups are implanted, so as to identify clusters of voxels of the image corresponding to the seed groups, respectively,
compute respective estimated positions of the brachytherapy seeds based on respective dimensions of each of the clusters, and
store or communicate the estimated positions for use in computing an effective dose of the brachytherapy seeds.

2. A method, comprising:
processing a three-dimensional image of a portion of a body of a subject in which multiple brachytherapy seeds grouped into one or more seed groups are implanted, so as to identify clusters of voxels of the image corresponding to the seed groups, respectively;
computing respective estimated positions of the brachytherapy seeds based on respective dimensions of each of the clusters; and
storing or communicating the estimated positions for use in computing an effective dose of the brachytherapy seeds.

3. The method according to claim 2, further comprising:
displaying at least part of the image with overlaid markers at the estimated positions so as to allow a user to adjust the estimated positions by performing an action selected from the group of actions consisting of: overlaying one or more additional markers, deleting one or more of the markers, and moving one or more of the markers; and
computing the effective dose based on the adjusted estimated positions.

4. The method according to claim 2, wherein processing the image comprises processing the image by applying a Bayesian Gaussian mixture model to the image.

5. The method according to claim 2, wherein processing the image comprises processing the image by applying a connected-components clustering algorithm to the image.

6. The method according to claim 2, wherein computing the respective estimated positions of the brachytherapy seeds comprises:
based on the respective dimensions of the clusters, computing respective estimated numbers of the brachytherapy seeds in the seed groups; and
computing the respective estimated positions based on the respective estimated numbers .

7. The method according to claim 6, wherein, for each of the clusters, computing the estimated number of those of the brachytherapy seeds in the seed group corresponding to the cluster comprises:
computing a length of a main axis of the cluster; and
computing the estimated number of the brachytherapy seeds in the seed group corresponding to the cluster based on the length.

8. The method according to claim 7, wherein, for each of the seed groups, computing the respective estimated positions of those of the brachytherapy seeds in the seed group comprises computing the estimated positions such that, per the estimated positions, respective sub-clusters of the voxels corresponding to those of the brachytherapy seeds in the seed group are distributed uniformly along the main axis.

9. The method according to claim 8, wherein, for each of the seed groups, computing the respective estimated positions of those of the brachytherapy seeds in the seed group comprises:
segmenting the main axis into the estimated number of equal-length segments; and
computing the estimated positions such that, per the estimated positions, the sub-clusters are aligned with the main axis and centered on the segments, respectively.

10. The method according to claim 2, wherein computing the estimated positions comprises computing the estimated positions by computing respective estimated center coordinates and estimated orientation vectors of the brachytherapy seeds.

11. The method according to claim 2, wherein computing the respective estimated positions of the brachytherapy seeds comprises:
receiving a total number of the brachytherapy seeds from a user; and
computing the respective estimated positions of the brachytherapy seeds based on the total number.

12. A computer software product comprising a tangible non-transitory computer-readable medium in which program instructions are stored, which instructions, when read by a processor, cause the processor to:
process a three-dimensional image of a portion of a body of a subject in which multiple brachytherapy seeds grouped into one or more seed groups are implanted, so as to identify clusters of voxels of the image corresponding to the seed groups, respectively,
compute respective estimated positions of the brachytherapy seeds based on respective dimensions of each of the clusters, and
store or communicate the estimated positions for use in computing an effective dose of the brachytherapy seeds.

13. The computer software product according to claim 12, wherein the instructions further cause the processor to:
display at least part of the image with overlaid markers at the estimated positions so as to allow a user to adjust the estimated positions by performing an action selected from the group of actions consisting of: overlaying one or more additional markers, deleting one or more of the markers, and moving one or more of the markers, and
compute the effective dose based on the adjusted estimated positions.

14. The computer software product according to claim 12, wherein the instructions cause the processor to process the image by applying a Bayesian Gaussian mixture model to the image.

15. The computer software product according to claim 12, wherein the instructions cause the processor to process the image by applying a connected-components clustering algorithm to the image.

16. The computer software product according to claim 12, wherein the instructions cause the processor to compute the respective estimated positions of the brachytherapy seeds by:
computing, based on the respective dimensions of the clusters, respective estimateed numbers of the brachytherapy seeds in the seed groups, and
computing the respective estimated positions based on the respective estimated numbers.

17. The computer software product according to claim 16, wherein, for each of the clusters, the instructions cause the processor to compute the estimated number of those of the brachytherapy seeds in the seed group corresponding to the cluster by:
computing a length of a main axis of the cluster, and
computing the estimated number of the brachytherapy seeds in the seed group corresponding to the cluster based on the length.

18. The computer software product according to claim 17, wherein, for each of the seed groups, the instructions cause the processor to compute the respective estimated positions of those of the brachytherapy seeds in the seed group such that, per the estimated positions, respective sub-clusters of the voxels corresponding to those of the brachytherapy seeds in the seed group are distributed uniformly along the main axis.

19. The computer software product according to claim 18, wherein, for each of the seed groups, the instructions cause the processor to compute the respective estimated positions of those of the brachytherapy seeds in the seed group by:
segmenting the main axis into the estimated number of equal-length segments, and
computing the estimated positions such that, per the estimated positions, the sub-clusters are aligned with the main axis and centered on the segments, respectively.

20. The computer software product according to claim 12, wherein the instructions cause the processor to compute the estimated positions by computing respective estimated center coordinates and estimated orientation vectors of the brachytherapy seeds.

21. The computer soft ware product according to claim 12, wherein the instructions cause the processor to compute the respective estimated positions of the brachytherapy seeds by:
receiving a total number of the brachytherapy seeds from a user, and
computing the respective estimated positions of the brachytherapy seeds based on the total number.
